(19) United States
(12) Patent Application Publication
(10) Pub. No.: US 2013/0035607 A1
(43) Pub. Date: Feb. 7, 2013

(54) IN-EAR INFORMATION ACQUIRING APPARATUS AND FIXING MECHANISM

(71) Applicants: Seiji WADA, Kanagawa (JP); Takeshi Asakawa, Chiba (JP); Naoya Sazuka, Tokyo (JP); Toshimitsu Tsuboi, Tokyo (JP); Akichika Tanaka, Chiba (JP)

(72) Inventors: Seiji WADA, Kanagawa (JP); Takeshi Asakawa, Chiba (JP); Naoya Sazuka, Tokyo (JP); Toshimitsu Tsuboi, Tokyo (JP); Akichika Tanaka, Chiba (JP)

(73) Assignee: Sony Corporation, Tokyo (JP)

(21) Appl. No.: 13/560,177

(22) Filed: Jul. 27, 2012

(30) Foreign Application Priority Data
Aug. 3, 2011 (JP) 2011-170033

(51) Int. Cl. A61B 5/01

(52) U.S. Cl. 600/549

(57) ABSTRACT

There is provided an in-ear information acquiring apparatus including: an information acquiring unit that is inserted in the ernal auditory canal and acquires information inside the ear; and a fixing mechanism that fixes the information acquiring unit inside the ernal auditory canal, wherein the fixing mechanism includes: a rod portion having a plurality of support portions, which are erected toward a wall surface of the ernal auditory canal, provided on an outer side surface thereof around an outer circumference thereof at at least one location along the ernal auditory canal; and an adjusting portion adjusting an erect state of the support portions provided on the rod portion.

### BACKGROUND

The present disclosure relates to an in-ear information acquiring apparatus and a fixing mechanism.

In recent years, a thermometer that measures body temperature by measuring radiant heat emitted from the ear drum has been proposed. With such a thermometer, a sensor that measures radiant heat from the eardrum is inserted via the external auditory canal and the radiant heat emitted from the ear drum is measured in a contactless manner. As one example, Japanese Patent No. 2,671,946 discloses an eardrum temperature measuring apparatus where a sensor unit, which is composed of a first temperature sensor that detects infrared radiation from the eardrum and generates an output voltage which is proportionate to a temperature difference between the ambient temperature and the eardrum temperature and a second temperature sensor that detects the temperature in the vicinity of the first temperature sensor, is inserted into the ernal auditory canal. The package that stores the sensor unit is held by a support member made of silicon rubber in a shape that substantially fills a gap between a first bent portion between the entrance to the ernal auditory canal and the ernal auditory canal itself, and by inserting the support member into the ernal auditory canal, the package can be positioned inside the ernal auditory canal.

To allow stable fixing inside the ernal auditory canal, the form of the inserted part of an existing thermometer that measures the radiant heat from the eardrum is normally formed in a shape that matches the ernal auditory canal (see for example Japanese Patent No. 2,671,946 and Japanese Laid-Open Patent Publication No. 2002-340681) or in the shape of a cone (see for example Japanese Laid-Open Patent Publication No. H11-28194).

### SUMMARY

However, the thermometers according to the cited patent documents have a premise of measuring radiant heat from the eardrum only once in a short time and do not consider a case where radiant heat is continuously measured over a long period. Since such thermometers will fall out of the ear if the inserted parts of such thermometers are not firmly pressed toward the eardrum at the entrance of the ernal auditory canal, it is difficult to keep the orientation of the sensor portions inserted inside the ernal auditory canal constant and such thermometers are very uncomfortable for the user.

For this reason, for a thermometer that measures radiant heat from the eardrum, it is desirable to stably orientate the sensor toward the eardrum and to keep the orientation of the sensor constant even during use over a long period.

According to an embodiment of the present disclosure, there is provided an in-ear information acquiring apparatus including an information acquiring unit that is inserted in the ernal auditory canal and acquires information inside the ear, and a fixing mechanism that fixes the information acquiring unit inside the ernal auditory canal. The fixing mechanism includes a rod portion having a plurality of support portions, which are erected toward a wall surface of the ernal auditory canal, provided on an outer side surface thereof around an outer circumference thereof at at least one location along the ernal auditory canal, and an adjusting portion adjusting an erect state of the support portions provided on the rod portion.

According to an embodiment of the present disclosure, there is provided a fixing mechanism including a rod portion having a plurality of support portions, which are erected toward a wall surface of the ernal auditory canal, provided on an outer side surface thereof around an outer circumference thereof at at least one location along the ernal auditory canal, and an adjusting portion adjusting an erect state of the support portions provided on the rod portion.

As described above, according to the embodiments of the present disclosure, a wire connected to an information acquiring unit is supported by a rod portion that has a plurality of support hairs provided around an outer circumference thereof. By doing so, the information acquiring unit is supported by the support hairs so as to be positioned in the center of the ernal auditory canal, which means that it is possible to stably orientate a sensor toward the eardrum and to keep the orientation of the sensor constant even during use over a long period.

As described above, according to the embodiments of the present disclosure, for a thermometer that measures radiant heat from the eardrum, it is possible to stably orientate a sensor toward the eardrum and to keep the orientation of the sensor constant even during use over a long period.

### BRIEF DESCRIPTION OF THE DRAWINGS

FIG. 1 is a diagram useful in explaining the overall configuration of an eardrum thermometer according to an embodiment of the present disclosure and shows a state where the eardrum thermometer is fixed inside the ernal auditory canal;

FIG. 2 is a diagram useful in explaining the overall configuration of the eardrum thermometer according to the same embodiment, and shows a state where the eardrum thermometer is capable of moving inside the ernal auditory canal;

FIG. 3 is a diagram useful in explaining another example configuration of a stopper;

FIG. 4 is a block diagram of a temperature measuring function of the eardrum thermometer according to the same embodiment;

FIG. 5 is a diagram useful in explaining the functional principles of a fixing mechanism according to the same embodiment;

FIG. 6 is a diagram useful in explaining a first example configuration of an ernal auditory canal-inserted part of the eardrum thermometer according to the same embodiment;

FIG. 7 is a cross-sectional view of the ernal auditory canal-inserted part in FIG. 6;

FIG. 8 is a diagram useful in explaining a modification to the configuration of the ernal auditory canal-inserted part shown in FIG. 6;

FIG. 9 is a diagram useful in explaining a second example configuration of the ernal auditory canal-inserted part of the eardrum thermometer according to the same embodiment;

FIG. 10 is a cross-sectional view showing the configuration of a rod portion of the ernal auditory canal-inserted part in FIG. 9;

FIG. 11 is a front view showing the configuration of a support hair unit of the ernal auditory canal-inserted part in FIG. 9 and shows a state when looking from a length direction of the rod portion in FIG. 9;

FIG. 12 is an exploded perspective view showing a third example configuration of the ernal auditory canal-inserted part of the eardrum thermometer according to the same embodiment;

FIG. 13 is a diagram useful in explaining a method of adjusting the erect state of support hairs in a fixing mechanism shown in FIG. 12;

FIG. 14 is a simplified perspective view showing one example configuration of an operation portion that operates the adjusting portion;

FIG. 15 is a simplified perspective view showing another example configuration of an operation portion that operates the adjusting portion;

FIG. 16 is a diagram useful in explaining an example arrangement of a plurality of support hairs in the fixing mechanism;

FIG. 17 is a diagram useful in explaining another example arrangement of the plurality of support hairs in the fixing mechanism;

FIG. 18 is a diagram useful in explaining the relationship between a distance detecting sensor and the eardrum according to the same embodiment;

FIG. 19 is a diagram useful in explaining the positional relationship between a received light intensity distribution detected by the distance detecting sensor and the position of the distance detecting sensor relative to the eardrum;

FIG. 20 is a functional block diagram showing the functional configuration of a distance detecting apparatus according to the same embodiment;

FIG. 21 is a flowchart showing a navigation process carried out by the distance detecting apparatus according to the same embodiment; and

FIG. 22 is a hardware configuration diagram showing an example hardware configuration of the distance detecting apparatus according to the same embodiment.

### DETAILED DESCRIPTION OF THE EMBODIMENT(S)

Hereinafter, preferred embodiments of the present disclosure will be described in detail with reference to the appended drawings. Note that, in this specification and the appended drawings, structural elements that have substantially the same function and structure are denoted with the same reference numerals, and repeated explanation of these structural elements is omitted.

The following description is given in the order indicated below.

### 1. Overall Configuration of Eardrum Thermometer

1-1. Overview of Eardrum Thermometer

1-2. Temperature Measurement Using Radiant Heat from Eardrum

### 2. Configuration and Function of Fixing Mechanism

2-1. Functional Principles of Fixing Mechanism

2-2. Configuration of Ernal Auditory Canal-Inserted Part of Eardrum Thermometer

2-3. Operation Portion for Operating Adjusting Portion

2-4. Function of Support Hairs

### 3. Detection of Distance Between Eardrum and Temperature Sensor

3-1. Overview of Distance Detecting Function

3-2. Functional Configuration of Distance Detecting Apparatus

3-3. Navigation by Distance Detecting Apparatus

### 4. Example Hardware Configuration

### 1. Overall Configuration of Eardrum Thermometer

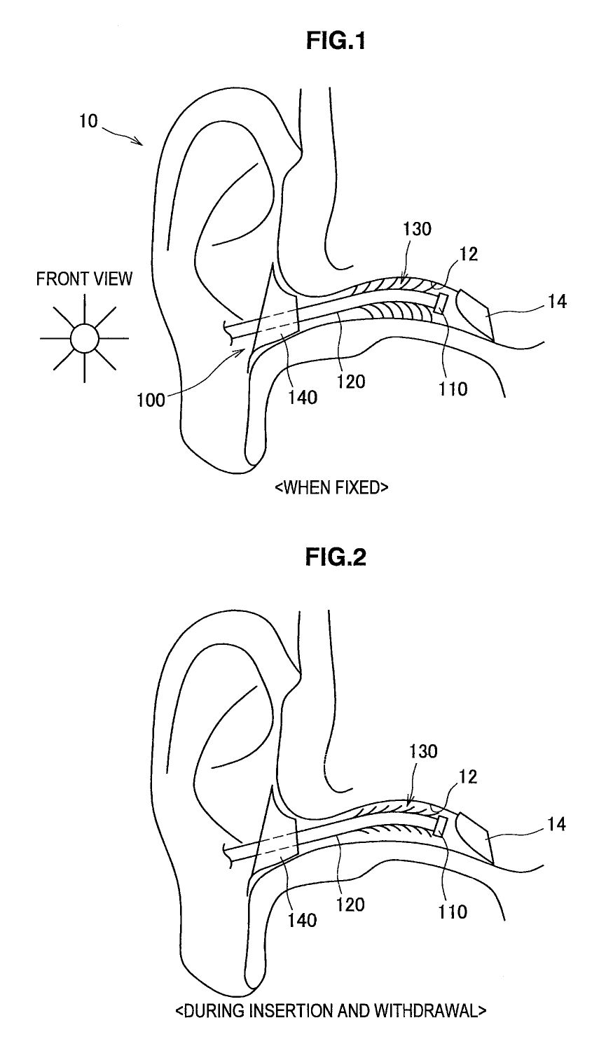

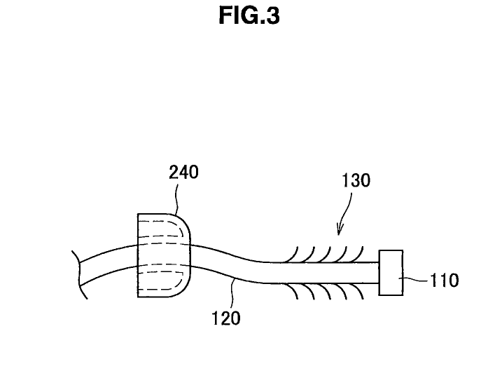

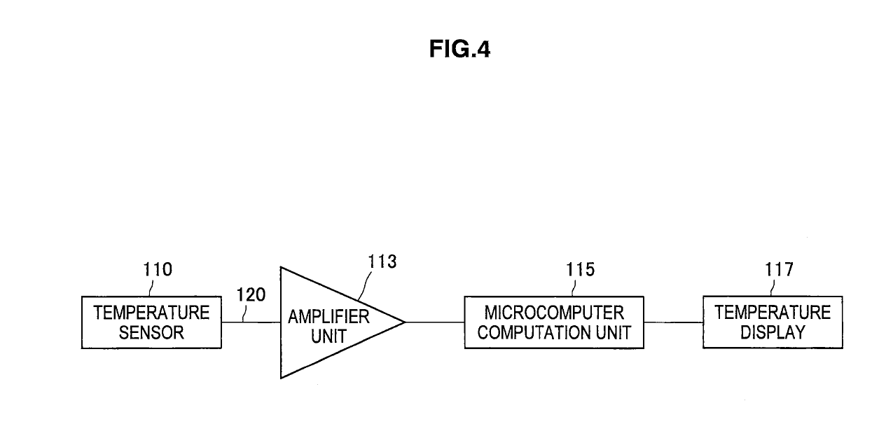

First, the overall configuration of an eardrum thermometer according to an embodiment of the present disclosure will be described with reference to FIGS. 1 to 4. FIG. 1 is a diagram useful in explaining the overall configuration of an eardrum thermometer 100 according to the present embodiment and shows a state where the eardrum thermometer 100 is fixed inside the ernal auditory canal 12. FIG. 2 is a diagram useful in explaining the overall configuration of the eardrum thermometer 100 according to the present embodiment and shows a state where the eardrum thermometer 100 is capable of moving inside the ernal auditory canal 12. FIG. 3 is a diagram useful in explaining another example configuration of a stopper. FIG. 4 is a block diagram of a temperature measuring function of the eardrum thermometer 100 according to the present embodiment.

1-1. Overview of Eardrum Thermometer

The eardrum thermometer according to the present embodiment is an in-ear information acquiring apparatus that measures body temperature by measuring radiant heat emitted from the eardrum as information inside the earhole. As shown in FIGS. 1 and 2, the eardrum thermometer 100 includes a temperature sensor 110 as an information acquiring unit that is inserted inside the ernal auditory canal 12 and acquires information inside the earhole and a sensor wire 120 that is connected to the temperature sensor 110 and extends toward the entrance of the ernal auditory canal. In addition, the eardrum thermometer 100 includes a fixing mechanism 130 that is provided on the outer circumference of the sensor wire 120 and fixes the temperature sensor 110 inside the ernal auditory canal 12 and a stopper 140 that restricts movement of the temperature sensor 110 in the direction of the eardrum 14.

When using the eardrum thermometer 100, the user places the temperature sensor 110 inside the ernal auditory canal 12 and inserts the temperature sensor 110 as far as the vicinity of the eardrum 14. When the temperature sensor 110 is inserted, the sensor wire 120 also becomes inserted inside the ernal auditory canal 12. The sensor wire 120 has a property whereby the sensor wire 120 can flexibly bend and is capable of being moved along the ernal auditory canal 12 which is curved. Note that when the temperature sensor 110 is moved inside the ernal auditory canal 12, the fixing mechanism 130 does not operate and the temperature sensor 110 can be moved freely.

Although the detailed configuration of the fixing mechanism 130 will be described later, when the temperature sensor 110 is moved inside the ernal auditory canal 12, as shown in FIG. 2, a plurality of support hairs that construct the fixing mechanism 130 do not contact the wall surface of the ernal auditory canal 12. By doing so, the movement of the temperature sensor 110 is not obstructed and the temperature sensor 110 can be moved inside the ernal auditory canal 12.

When the temperature sensor 110 is at an appropriate position where direct measurement of radiant heat from the eardrum 14 is possible, as shown in FIG. 1, the fixing mechanism 130 can be operated to fix the temperature sensor 110 at such appropriate position. Although the detailed configuration of the fixing mechanism 130 will be described later, by having the front ends of the plurality of support hairs, which are erected toward the wall surface of the ernal auditory canal 12 from the outer circumference of the sensor wire 120, contact the wall surface of the ernal auditory canal 12, resistance is produced when the temperature sensor 110 tries to move toward the eardrum 14. By doing so, it is possible to fix the position of the temperature sensor 110.

The eardrum thermometer 100 is configured so that when the temperature sensor 110 is positioned at an appropriate position inside the ernal auditory canal 12, the stopper 140 catches on the entrance of the ernal auditory canal 12 and the temperature sensor 110 becomes unable to move further toward the eardrum 14. In this way, through the functioning of the fixing mechanism 130 and the stopper 140, it is possible to prevent the temperature sensor 110 from moving from the appropriate position toward the eardrum 14 and thereby ensure the safety of the user. Note that although the stopper 140 shown in FIG. 1 is formed in a shape with a brim that is larger than the area of the entrance of the ernal auditory canal 12 so that the stopper 140 catches on the entrance of the ernal auditory canal 12, the present disclosure is not limited to this example and as one example it is also possible to use a stopper 240 in the form of an ear pad as shown in FIG. 3.

When, the temperature sensor 110 is pulled out toward the entrance of the ernal auditory canal 12 from a state shown in FIG. 1 where the eardrum thermometer 100 is fixed at the appropriate position, the fixing mechanism 130 is released once again. By doing so, the plurality of support hairs that contacted the wall surface of the sensor wire 120 are placed in a non-contact state as shown in FIG. 2 and it becomes possible for the temperature sensor 110 to move. Note that by providing the stopper 140 as described above, the temperature sensor 110 will not move toward the eardrum 14 beyond the appropriate position, so that even if the fixing mechanism 130 is released, the temperature sensor 110 will not accidently move toward the eardrum 14.

1-2. Temperature Measurement Using Radiant Heat from Eardrum

Next, the temperature measuring function of the eardrum thermometer 100 according to the present embodiment will be described with reference to FIG. 4. As shown in FIG. 4, in the eardrum thermometer 100 according to the present embodiment, a temperature measuring function is realized by the temperature sensor 110, an amplifier unit 113, a microcomputer computation unit 115, and a temperature display 117.

The temperature sensor 110 is a sensor that measures the temperature of the eardrum 14 by measuring radiant heat from the eardrum 14. As shown in FIG. 1 and FIG. 2, the temperature sensor 110 is placed inside the ernal auditory canal 12 and is inserted as far as the appropriate position where the radiant heat from the eardrum 14 can be detected. The temperature sensor 110 according to the present embodiment can be constructed for example from an infrared sensor (such as a thermopile composed of a plurality of thermocouples) that detects infrared radiation from the eardrum 14 and a temperature compensating sensor (such as a thermistor or a diode). The temperature sensor 110 converts the heat energy of the measured radiant heat to a voltage and outputs the voltage via the sensor wire 120 to the amplifier unit 113.

The amplifier unit 113 amplifies the voltage inputted from the temperature sensor 110, converts the result to a digital signal, and outputs the digital signal to the microcomputer computation unit 115.

The microcomputer computation unit 115 calculates the temperature of the eardrum 14 based on the digital signal inputted from the amplifier unit 113. The microcomputer computation unit 115 calculates the temperature difference between the temperature of the eardrum 14 and the ambient temperature of the infrared sensor from a digital signal corresponding to measurements made by the infrared sensor and also calculates the ambient temperature of the infrared sensor from a digital signal corresponding to a measurement made by the temperature compensating sensor. The microcomputer computation unit 115 then calculates the temperature of the eardrum 14 by correcting the ambient temperature of the infrared sensor using the temperature difference between the ambient temperature and the temperature of the eardrum 14. The temperature of the eardrum 14 calculated by the microcomputer computation unit 115 is outputted to the temperature display 117 and is displayed on the temperature display 117.

By being equipped with this temperature measuring function, the eardrum thermometer 100 is capable of measuring the temperature of the eardrum 14. Note that as described later, out of the temperature measuring function of the eardrum thermometer 100, it is sufficient for at least the temperature sensor 110 to be capable of insertion inside the ernal auditory canal 12. The amplifier unit 113, the microcomputer computation unit 115 and the temperature display 117 may all be provided outside the ernal auditory canal 12.

### 2. Configuration and Function of Fixing Mechanism

The eardrum thermometer 100 according to the present embodiment is equipped with the fixing mechanism 130 that can flex and thereby match the curves of the ernal auditory canal 12 and can stably place the temperature sensor 110 facing the eardrum 14. The configuration and function of the fixing mechanism 130 will now be described in detail.

2-1. Functional Principles of Fixing Mechanism

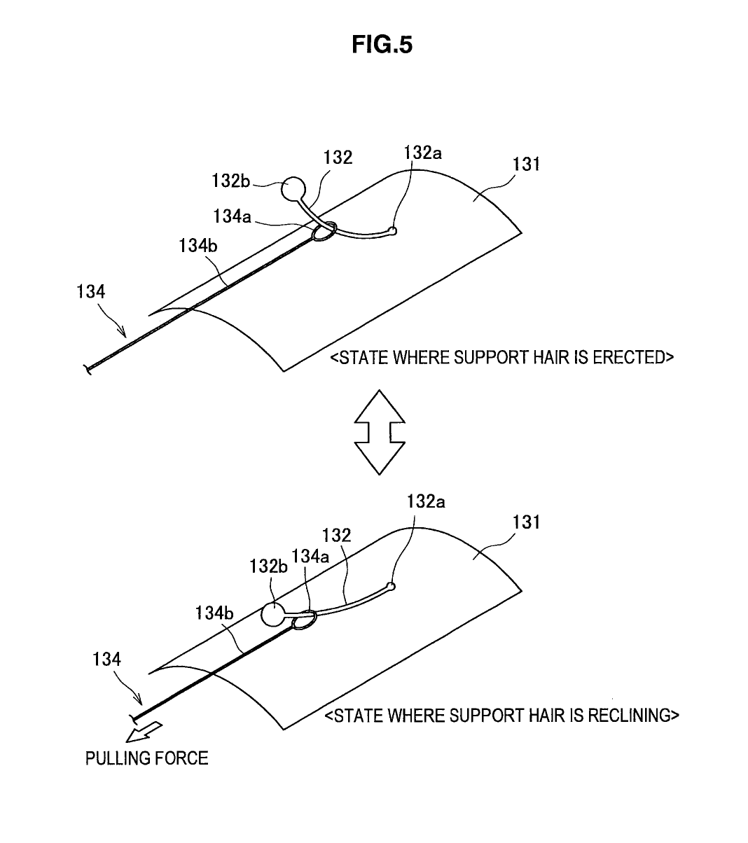

First, the functional principles of the fixing mechanism 130 according to the present embodiment will be described with reference to FIG. 5. FIG. 5 is a diagram useful in explaining the functional principles of the fixing mechanism 130 according to the present embodiment.

The fixing mechanism 130 is composed of a plurality of support hairs 132 provided on a rod portion 131 that covers the outer circumference of the sensor wire 120 and an adjusting portion 134 that changes the erection angle of the support hairs 132 to adjust the erect state of the support hairs 132. FIG. 5 shows an enlargement of one support hair 132, out of the plurality of support hairs 132 that construct the fixing mechanism 130.

The support hairs 132 are made of a material such as nylon, polyethylene, or polypropylene, and are provided so that an erect state where the support hairs 132 are bent back and upward from the surface of the rod portion 131 is a base state. The support hairs 132 are set with a length that contacts the wall surface of the ernal auditory canal 12 in a state where the support hairs 132 are erected inside the ernal auditory canal 12. Such length is set at around 5 to 10 mm, for example. As one example, front ends 132b of the support hairs 132 may be spherical as shown in FIG. 5. By doing so, since the front ends 132b of the support hairs 132 will make point contact when contacting the wall surface of the ernal auditory canal 12, it is possible for the plurality of support hairs 132 to uniformly apply a load to the wall surface of the ernal auditory canal 12. Also, by making the front ends 132b of the support hairs 132 spherical, it is possible to increase safety when the support hairs 132 contact the wall surface of the ernal auditory canal 12 and to reduce discomfort.

The support hairs 132 are provided on the eardrum thermometer 100 so as to be bent from support points 132a where the support hairs 132 are fixed to the rod portion 131 to the front ends 132b in a direction that obstructs movement of the temperature sensor 110 from the entrance of the ernal auditory canal 12 toward the eardrum 14. By doing so, when the support hairs 132 are in the erect state, the support hairs 132 contact the wall surface of the ernal auditory canal 12 and prevent the temperature sensor 110 from moving toward the eardrum 14. Meanwhile, if the support hairs 132 are prevented from contacting the wall surface of the ernal auditory canal 12 when the temperature sensor 110 has been inserted, it is possible for the temperature sensor 110 to be inserted inside the ernal auditory canal 12 and approach the eardrum 14. To switch between the contact and non-contact states of the support hairs 132 on the wall surface of the ernal auditory canal 12 in this way, the adjusting portion 134 for changing the erection angle of the support hairs 132 relative to the surface of the rod portion 131 is provided on the fixing mechanism 130.

As shown in FIG. 5, for example, the adjusting portion 134 is composed of a ring portion 134a, through which a support hair 132 is inserted, and a pullstring 134b connected to the ring portion 134a. As shown at the top in FIG. 5, when the adjusting portion 134 is maintaining the basic state of a support hair 132, the support hair 132 is in a standing state (hereinafter the “erect state”). Meanwhile, when a pulling force in the direction where the support hair 132 is bent back is applied to the pullstring 134b of the adjusting portion 134, the ring portion 134a connected to the pullstring 134b is moved toward the front end 132b of the support hair 132. By doing so, as shown at the bottom in FIG. 5, the support hair 132 is placed in a reclining state where the front end 132b approaches the rod portion 131 and the erection angle is small compared to the basic state.

That is, when a pulling force is applied to the pullstring 134b of the adjusting portion 134, the support hair 132 is placed in the reclining state, thereby making it possible to remove the contact with the wall surface of the ernal auditory canal 12. Meanwhile, when the pulling force on the pullstring 134b of the adjusting portion 134 is removed, the support hair 132 is placed in the erect state, thereby making it possible to contact the wall surface of the ernal auditory canal 12.

2-2. Configuration of Ernal Auditory Canal-Inserted Part of Eardrum Thermometer

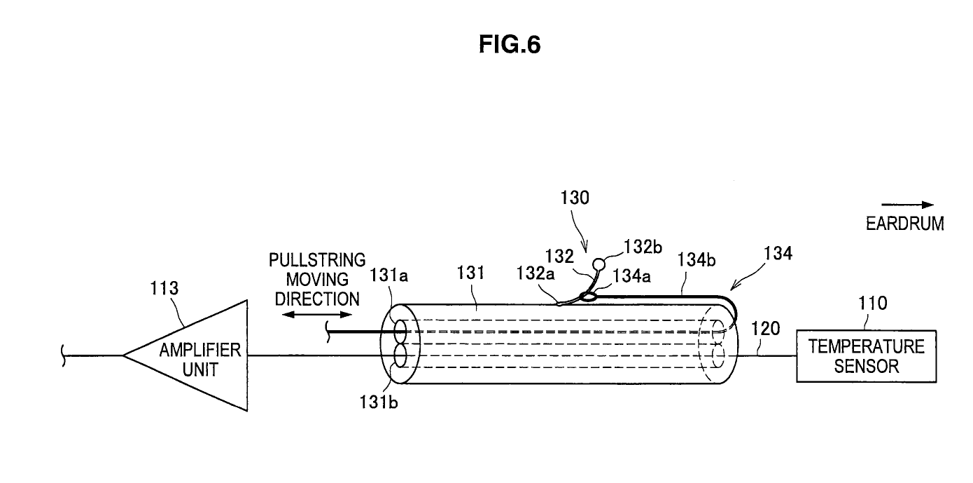

The configuration of a part of the eardrum thermometer 100 according to the present embodiment that is inserted into the ernal auditory canal 12 and is configured based on the functional principles of the fixing mechanism 130 described above will now be described with reference to FIGS. 6 to 13.

### (a) First Example Configuration

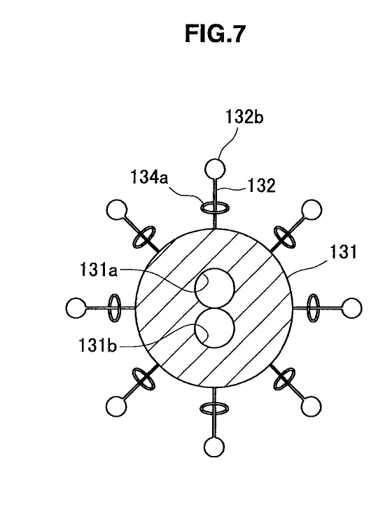

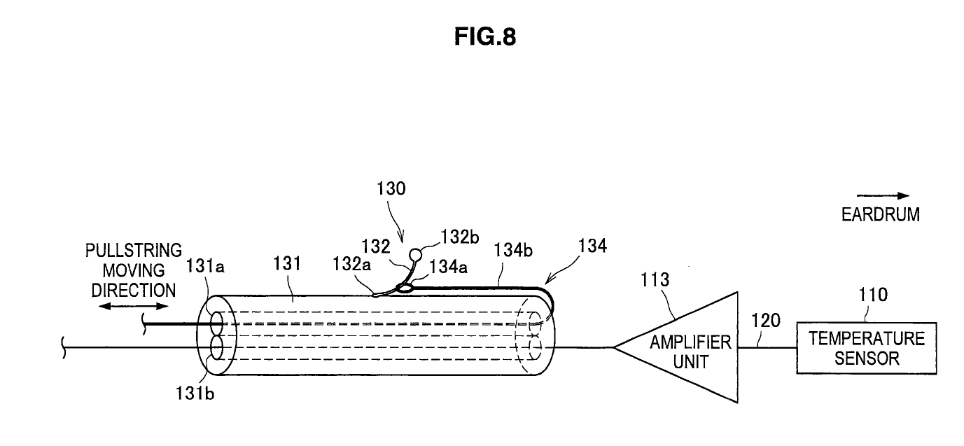

First, a first example configuration of the ernal auditory canal-inserted part of the eardrum thermometer 100 according to the present embodiment will be described with reference to FIGS. 6 to 8. FIG. 6 is a diagram useful in explaining a first example configuration of an ernal auditory canal-inserted part of the eardrum thermometer 100 according to the present embodiment. FIG. 7 is a cross-sectional view of the ernal auditory canal-inserted part in FIG. 6. FIG. 8 is a diagram useful in explaining a modification to the configuration of the ernal auditory canal-inserted part shown in FIG. 6. Note that in FIGS. 6 to 8, for ease of explanation, the sensor wire 120 is shown as a straight wire and the rod portion 131 of the fixing mechanism 130 is enlarged. The external form of the eardrum thermometer 100 is the shape shown in FIGS. 1 and 2. Note that although only one support hair 132 is shown in FIGS. 6 and 8 for ease of explanation, a plurality of the support hairs 132 are provided on the rod portion 131.

In the first configuration example of the ernal auditory canal-inserted part of the eardrum thermometer 100, as shown in FIGS. 6 and 7, the fixing mechanism 130 is provided between the temperature sensor 110 and the amplifier unit 113 that construct the temperature measuring function described with reference to FIG. 4 and are connected by the sensor wire 120. A first passage 131a and a second passage 131b that pass through in the length direction are formed in the rod portion 131 of the fixing mechanism 130. In the present embodiment, as described later the pullstring 134b of the adjusting portion 134 is inserted through the first passage 131a and the sensor wire 120 is inserted through the second passage 131b. That is, the ernal auditory canal-inserted part of the eardrum thermometer 100 and has the temperature sensor 110 disposed at the front end (relative to the fixing mechanism 130) that is inserted into the ernal auditory canal 12, with the sensor wire 120 that is connected to the temperature sensor 110 and the pullstring 134b extending from the opposite side to the temperature sensor 110.

Note that although the fixing mechanism 130 is provided between the temperature sensor 110 and the amplifier unit 113 in FIG. 6, the present disclosure is not limited to this example and as shown in FIG. 8 for example, the amplifier unit 113 may be provided on the temperature sensor 110 side of the fixing mechanism 130. In such case, the sensor wire 120 that connects the amplifier unit 113 and the microcomputer computation unit 115 passes through the first passage of the rod portion 131.

The plurality of support hairs 132 are provided on the outer circumferential surface of the rod portion 131 of the fixing mechanism 130. The support hairs 132 are provided so as to be bent back and upward toward the eardrum 14, that is, bent back and upward toward the side where the temperature sensor 110 is provided. By doing so, when the support hairs 132 are in the erect state, it becomes no longer possible for the temperature sensor 110 to move inside the ernal auditory canal 12 toward the eardrum 14.

The adjusting portion 134 is provided on each of the support hairs 132. Each adjusting portion 134 is composed of the ring portion 134a through which a support hair 132 passes and the pullstring 134b that is connected to the ring portion 134a. Here, to enable the pulling force applied to the pullstring 134b to be adjusted outside the ernal auditory canal 12, the pullstring 134b extends from the ring portion 134a toward the temperature sensor 110 and is then pulled out toward the opposite side to the temperature sensor 110 through the first passage 131a of the rod portion 131.

When a pulling force has been applied to the pullstring 134b toward the opposite side to the temperature sensor 110, the ring portion 134a moves toward the temperature sensor 110. That is, the ring portion 134a moves toward the front end 132b of the support hair 132 inserted through the ring portion 134a. By doing so, the erection angle of the support hair 132 that was erected toward the temperature sensor 110 falls and the support hair 132 is placed in the reclining state. Meanwhile, when the pulling force applied to the pullstring 134b in the opposite direction to the temperature sensor 110 is relaxed, the ring portion 134a moves in the opposite direction to the temperature sensor 110 according to a restorative force whereby the support hair 132 tries to return to the base state. That is, the ring portion 134a moves toward the support point 132a of the support hair 132 passed through the ring portion 134a. By doing so, it is possible for the support hair 132 that was in the reclining state to be placed in the erect state toward the temperature sensor 110.

By doing so, by applying a pulling force to the pullstrings 134b from outside the ernal auditory canal 12 and relaxing the applied pulling force, it is possible for the user to adjust the erect state of the support hairs 132.

### (b) Second Example Configuration

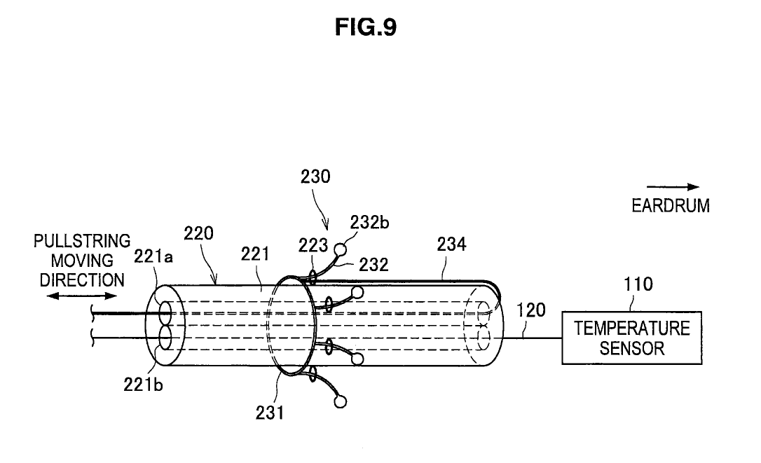

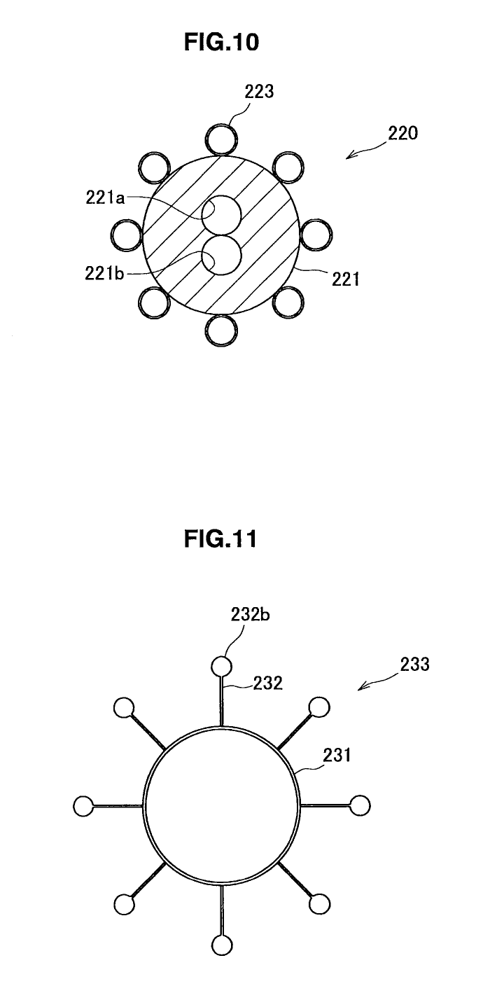

Next, a second example configuration of the ernal auditory canal-inserted part of the eardrum thermometer 100 according to the present embodiment will be described with reference to FIGS. 9 to 11. FIG. 9 is a diagram useful in explaining this second example configuration of the ernal auditory canal-inserted part of the eardrum thermometer 100 according to the present embodiment. FIG. 10 is a cross-sectional view showing the configuration of a rod portion 220 of the ernal auditory canal-inserted part in FIG. 9. FIG. 11 is a front view showing the configuration of a support hair unit 233 of the ernal auditory canal-inserted part in FIG. 9 and shows a state when looking from the length direction of the rod portion 220 in FIG. 9. Note that in FIG. 9, for ease of explanation, the sensor wire 120 is shown as a straight wire and the rod portion 220 of a fixing mechanism 230 is enlarged. The external form of the eardrum thermometer 100 is the shape shown in FIGS. 1 and 2.

In this second configuration example of the ernal auditory canal-inserted portion of the eardrum thermometer 100, as shown in FIG. 9, the fixing mechanism 230 is provided between the temperature sensor 110 and the amplifier unit 113 that construct the temperature measuring function described with reference to FIG. 4 and are connected by the sensor wire 120. A first passage 231a and a second passage 231b that pass through in the length direction are formed in the rod portion 220 of the fixing mechanism 230. In the present embodiment, as described later the pullstring 234 of an adjusting portion is inserted through the first passage 221a and the sensor wire 120 is inserted through the second passage 221b. That is, the ernal auditory canal-inserted part of the eardrum thermometer 100 has the temperature sensor 110 disposed at the front end (relative to the fixing mechanism 230) that is inserted into the ernal auditory canal 12 and the sensor wire 120 that is connected to the temperature sensor 110 and the pullstring 234 extend from the opposite side to the temperature sensor 110. Note that as described earlier for the first example configuration, the amplifier unit 113 may be provided on the temperature sensor 110-side of the fixing mechanism 230.

Also, as shown in FIG. 10, a plurality of ring portions 223 are fixed to the outer circumferential surface of the rod portion 220. As shown in FIG. 9, support hairs 232 of the support hair unit 233 described later are respectively inserted through the individual ring portions 223.

The support hair unit 233 composed of the plurality of support hairs 232 is provided on the outer circumferential surface of the rod portion 220. Note that although a case where one support hair unit 233 is provided on the fixing mechanism 230 is shown in FIG. 9, the present disclosure is not limited to this example and a plurality of support hair units 233 may be provided on the fixing mechanism 230.

As shown in FIG. 11, the support hair unit 233 is configured with the plurality of support hairs 232 radially extending from a ring 231 through which the rod portion 220 is inserted. The support hair unit 233 is provided so as to be capable of moving relative to the rod portion 220 in the length direction of the rod portion 220. Here, the support hairs 232 are provided so as to be bent back and upward toward the eardrum 14, or in other words bent back and upward toward the side where the temperature sensor 110 is provided. By doing so, when the support hairs 232 are in the erect state, it becomes no longer possible inside the ernal auditory canal 12 for the temperature sensor 110 to move toward the eardrum 14.

The pullstring 234 is provided on each support hair unit 233 as an adjusting portion. As shown in FIG. 9, one end of the pullstring 234 is fixed to the ring 231 of the support hair unit 233. To enable the pulling force applied to the pullstring 234 to be adjusted outside the ernal auditory canal 12, the pullstring 234 extends from the ring 231 toward the temperature sensor 110 and is then pulled out in the opposite direction to the temperature sensor 110 through the first passage 221a of the rod portion 220.

If a pulling force is applied to the pullstring 234 toward an opposite side to the temperature sensor 110, the ring moves toward the temperature sensor 110. By doing so, the support hairs 232 fixed to the ring 231 also move toward the temperature sensor 110. At this time, since the ring portions 223 fixed to the rod portion 220 are positioned at the bases (that is, the parts fixed to the ring 231) of the support hairs 232, the support hairs 232 are placed in the erect state. Meanwhile, when the pulling force toward the opposite side to the temperature sensor 110 that was applied to the pullstring 234 is relaxed, the ring 231 moves in the opposite direction to the temperature sensor 110. By doing so, the support hairs 232 fixed to the ring 231 also move in the opposite direction to the temperature sensor 110. At this time, since the ring portions 223 fixed to the rod portion 220 are positioned at front ends 232b of the support hairs 232, the erection angle of the support hairs 232 that were erected toward the temperature sensor 110 falls and the support hairs 232 are placed in the reclining state.

In this way, by applying a pulling force to the pullstring 234 and easing the applied pulling force outside the ernal auditory canal 12, the user is capable of adjusting the erect state of the support hairs 232.

Note that although the pullstring 234 that is the adjusting portion extends from the ring 231 toward the temperature sensor 110 and is then pulled out in the opposite direction to the temperature sensor 110 through the first passage 221a of the rod portion 220 in FIG. 9, the present disclosure is not limited to this example. For example, a through-hole that passes through to the first passage 221a may be formed in the outer circumference of the rod portion 220 at a position that is even further from the temperature sensor 110 than a position where the ring 231 is located furthest from the temperature sensor 110. The pullstring 234 passes from the ring 231 through such through-hole and the first passage 221a and is pulled out in the opposite direction to the temperature sensor 110. With this configuration, if a pulling force is applied to the pullstring 234 in the opposite direction to the temperature sensor 110, the ring 231 moves in the opposite direction to the temperature sensor 110 and the support hairs 232 are placed in the reclining state. Meanwhile, if the pulling force applied to the pullstring 234 in the opposite direction to the temperature sensor 110 is relaxed, the ring 231 moves in the direction toward the temperature sensor 110 and the support hairs 232 that were in the reclining state are erected toward the temperature sensor 110.

### (c) Third Example Configuration

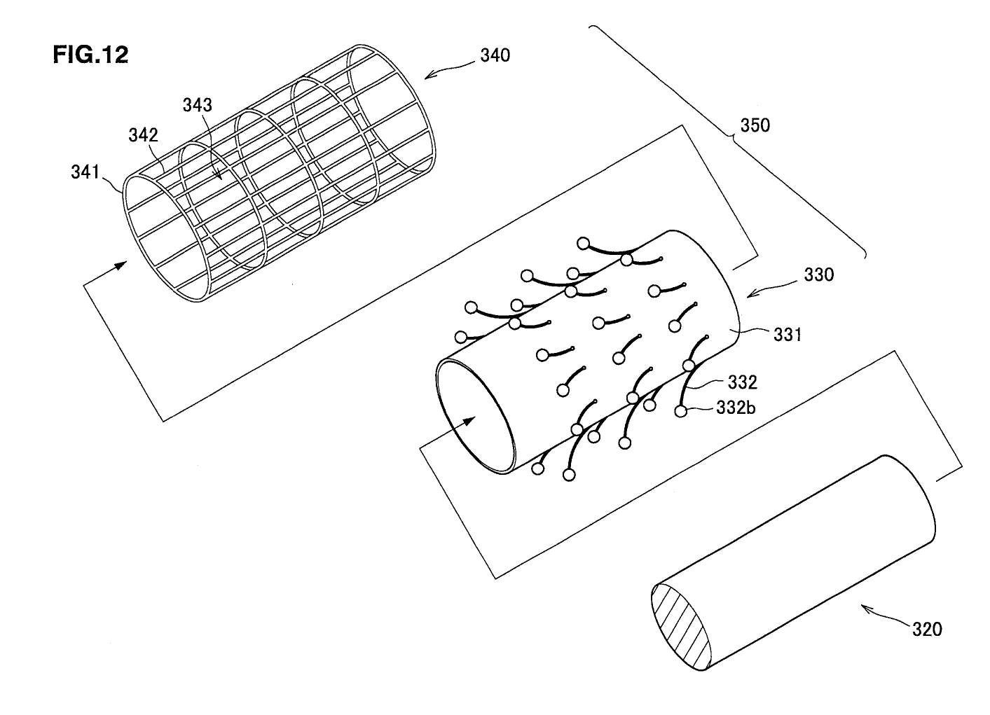

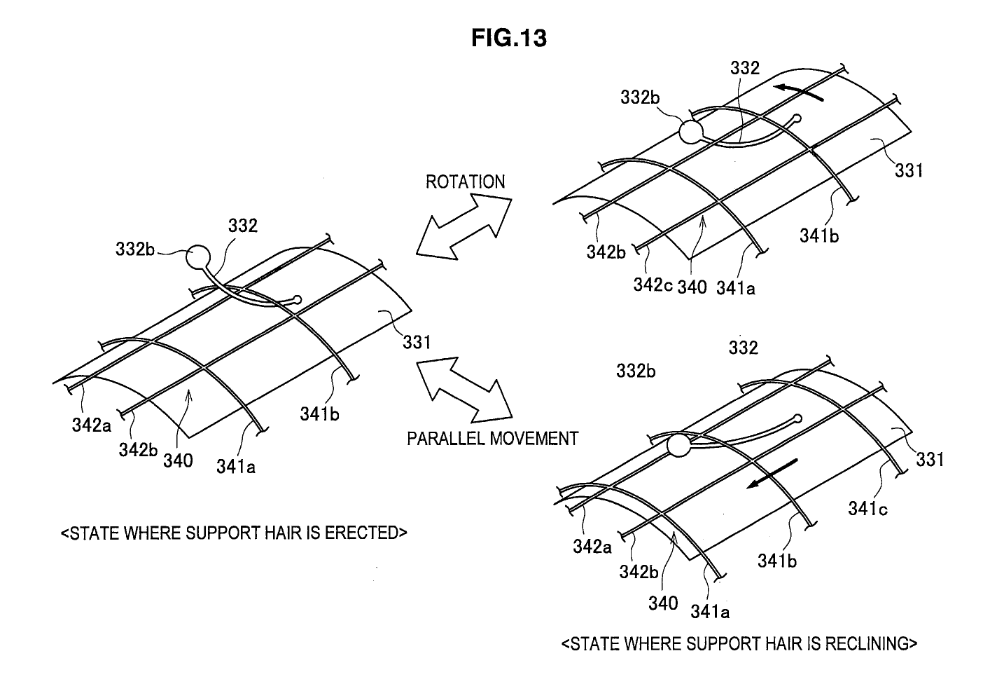

Next, a third example configuration of the ernal auditory canal-inserted part of the eardrum thermometer 100 according to the present embodiment will be described with reference to FIGS. 12 and 13. FIG. 12 is an exploded perspective view showing this third example configuration of an ernal auditory canal-inserted part of the eardrum thermometer 100 according to the present embodiment. FIG. 13 is a diagram useful in explaining a method of adjusting the erect state of support hairs 332 in the fixing mechanism shown in FIG. 12. Note that although only part of a sensor wire 320 is shown in FIG. 12, in reality the sensor wire 320 extends in the length direction as shown in FIG. 1 and FIG. 2, with the temperature sensor 110 being provided at one end of the sensor wire 320 and the other end of the sensor wire 320 extending outside the ernal auditory canal 12.

In this third example configuration of the ernal auditory canal-inserted part of the temperature sensor 110, as shown in FIG. 12, the fixing mechanism 350 is provided between the temperature sensor 110 and the amplifier unit 113 that construct the temperature measuring function described with reference to FIG. 4 and are connected by the sensor wire 320 (that corresponds to numeral 120 in FIG. 4). The fixing mechanism 350 is composed of a hollow rod portion 330, which covers the sensor wire 320 and is fixed to the sensor wire 320, and a mesh unit 340, which is inserted onto the outer circumference of the rod portion 330.

More specifically, the rod portion 330 is fixed to the sensor wire 320 in the ernal auditory canal-inserted part of the eardrum thermometer 100. The plurality of support hairs 332 are provided on an outer circumferential surface 331 of the rod portion 330. The support hairs 332 are provided so as to be bent back and upward toward the eardrum 14, that is, bent back and upward toward the side where the temperature sensor 110 is provided. By doing so, when the support hairs 232 are in the erect state, inside the ernal auditory canal 12 it becomes no longer possible for the temperature sensor 110 to move toward the eardrum 14. The support hairs 332 are inserted through openings 343 of the mesh unit 340.

As shown in FIG. 12, the mesh unit 340 is an adjusting portion that includes a plurality of openings 343 through which the support hairs 332 of the rod portion 330 are inserted. As one example, the mesh unit 340 can be constructed by fixing together a plurality of rings 341 that are concentrically aligned using a plurality of bars 342 that extend in the direction of alignment of the rings 341 and are disposed in the circumferential direction of the rings 341. Note that the mesh unit 340 is not limited to the example shown in FIG. 12 and as one example can also be formed by providing a plurality of through-holes in the outer circumferential surface of a tube-shaped member. The mesh unit 340 is inserted onto the outer circumference of the rod portion 330 so as to be capable of at least one of moving parallel to the length direction of the sensor wire 320 and rotationally moving in the circumference direction of the sensor wire 320.

In this third example configuration, the erect state of the support hairs 332 of the rod portion 330 can be adjusted by moving the mesh unit 340 relative to the rod portion 330 fixed to the sensor wire 320. First, when the support hairs 332 are erected, the mesh unit 340 is disposed so that the rings 341 or bars 342 that construct the openings 343 are positioned on front end sides 332b of the support hairs 332 so as to not reduce the erection angle. For example, as shown on the left in FIG. 13, the mesh unit 340 is disposed so that the bars 342a, 342b do not contact a support hair 332 and the ring 341b is positioned at the base (the part fixed to the rod portion 330) of the support hair 332. By doing so, it is possible to maintain the basic state where the support hair 332 is erected.

Meanwhile, by operating the mesh unit 340 to position the ring 341 or the bars 342 that construct the opening 343 on the front end side 332b of the support hair 332, the support hair 332 is placed in the reclining state. First consider a case where the mesh unit 340 operates by moving in parallel to the length direction of the sensor wire 320. For example, if the mesh unit 340 in the state shown on the left in FIG. 13 moves in parallel with a specified direction (here, the direction where the ring 341b approaches the ring 341a side), the support hair 332 will be pressed by the ring 341b as far as the front end 332b as shown in the lower right part of FIG. 13. By doing so, the erection angle of the support hair 332 decreases and the support hair 332 is placed in the reclining state.

Next, consider a case where the mesh unit 340 operates by rotationally moving in the circumference direction of the sensor wire 320. For example, if the mesh unit 340 in the state shown on the left in FIG. 13 is rotated in the anti-clockwise direction, as shown in the upper right part of FIG. 13, the support hair 332 will be pressed by the bar 342b as far as the vicinity of the front end 332b. By doing so, the erection angle of the support hair 332 falls and the support hair 332 is placed in the reclining state.

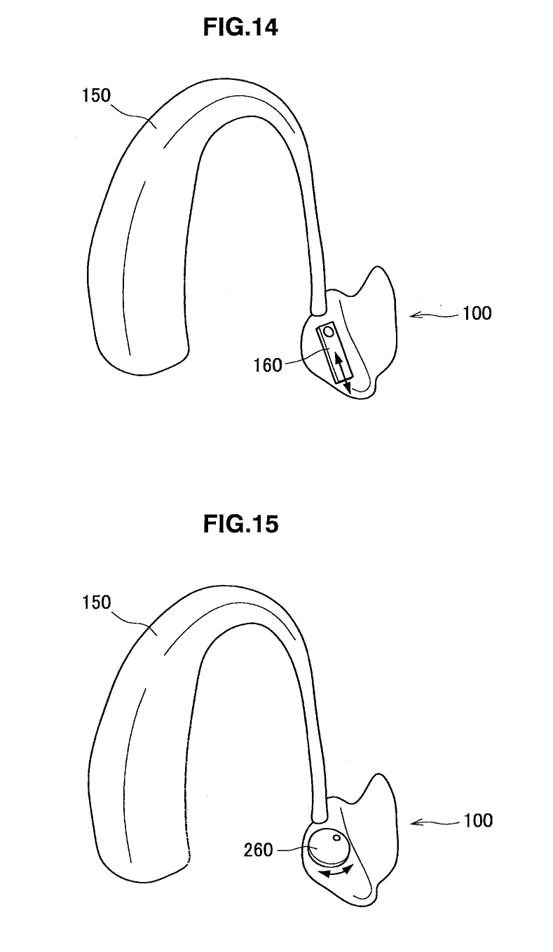

After the mesh unit 340 has been operated and the support hair 332 has been placed in the reclining state, to place the support hair 332 back in the erect state, an opposite operation to that described above may be performed. By doing so, the support hair 332 stops being pressed by the ring 341 or the bar 342 that construct the mesh unit 340 and the released support hair 332 is placed in the erected base state due to the restorative force. Note that the mesh unit 340 is capable of being operated by operating an operation portion (numeral 160 in FIG. 14 or number 260 in FIG. 15), described later. In this way, by operating the operation portion outside the ernal auditory canal 12, the user is capable of adjusting the erect state of the support hairs 332.

2-3. Operation Portion for Operating Adjusting Portion

The eardrum thermometer 100 according to the present embodiment includes an operation portion that operates the respective adjusting portions of the fixing mechanisms described earlier to enable the adjusting portions to be easily operated by the user outside the ernal auditory canal 12. Example configurations of the operation portion are shown in FIG. 14 and FIG. 15. FIG. 14 and FIG. 15 are perspective views showing an attachment portion 150 for attaching the eardrum thermometer 100 to the ear 10. The attachment portion 150 is formed in a shape that is curved so as to be capable of hooking onto the ear 10 as shown in FIG. 14 and FIG. 15. At one end of the attachment portion 150, an ernal auditory canal-inserted part of the eardrum thermometer 100 shown in FIG. 1 and FIG. 2 is provided. The adjusting portion that extends from the ernal auditory canal-inserted part outside the ernal auditory canal 12 is connected to the operation portion (numerals 160, 260) provided outside the ernal auditory canal 12.

As one example, the operation portion can be configured as a slide operation portion 160 that moves in one direction as shown in FIG. 14. When the slide operation portion 160 is moved in a first direction, the adjusting portion functions in keeping with the movement of the slide operation portion 160 and is capable of causing the support hairs to recline inside the ernal auditory canal 12. Meanwhile, when the slide operation portion 160 is moved in a second direction that is the opposite to the first direction, the adjusting portion functions in keeping with the movement of the slide operation portion 160 and is capable of erecting the support hairs inside the ernal auditory canal 12.

As another example, the operation portion can be configured as the rotational operation portion 260 that rotates as shown in FIG. 15. When the rotational operation portion 260 is rotated in a first direction, the adjusting portion functions in keeping with the movement of the rotational operation portion 260 and is capable of causing the support hairs to recline inside the ernal auditory canal 12. Meanwhile, when the rotational operation portion 260 is rotated in a second direction that is the opposite to the first direction, the adjusting portion functions in keeping with the movement of the rotational operation portion 260 and is capable of erecting the support hairs inside the ernal auditory canal 12.

Such operation portions are capable of being manually operated by the user and are also capable of being operated by driving a driving unit such as a motor according to operation instructions from the eardrum thermometer 100.

2-4. Function of Support Hairs

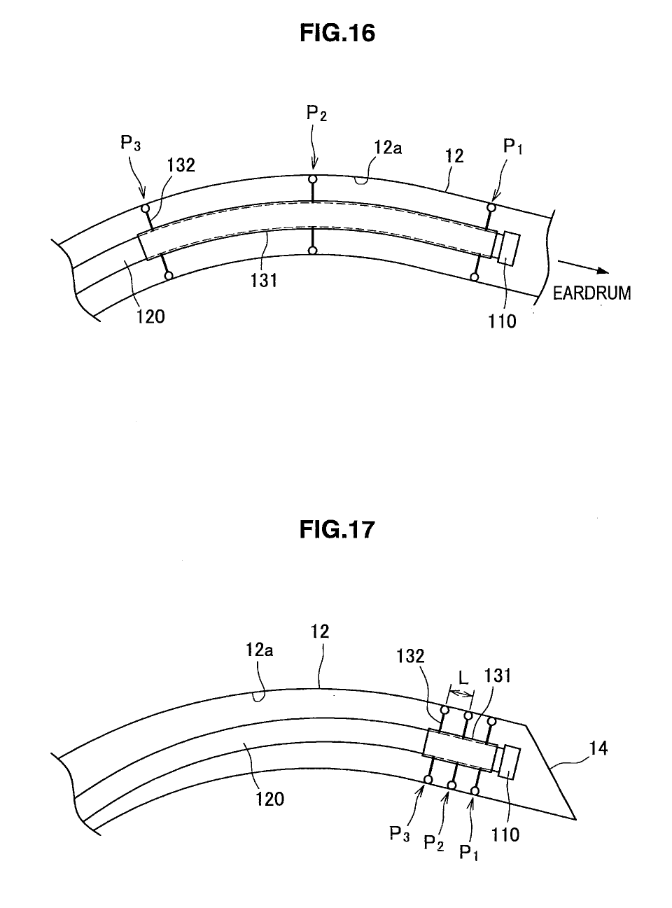

Next, the function of the support hairs according to the present embodiment will be described in detail with reference to FIG. 16 and FIG. 17. FIG. 16 is a diagram useful in explaining one example arrangement of the plurality of support hairs 132 on the fixing mechanism 130. FIG. 17 is a diagram useful in explaining a different example arrangement of the plurality of support hairs 132 on the fixing mechanism 130.

In the erect state, the support hairs 132 according to the present embodiment contact the wall surface 12a of the ernal auditory canal 12 and function as a stopper that holds the temperature sensor 110 so as to face the eardrum 14 in an optimal state for measuring radiant heat from the eardrum 14. Due to the direction in which the support hairs 132 are bent upward and back, the support hairs 132 also function so as to obstruct movement of the temperature sensor 110 in a specified direction.

To position the temperature sensor 110 facing the eardrum 14 in an optimal state, on the eardrum thermometer 100 according to the present embodiment, the plurality of support hairs 132 are disposed in the circumferential direction at at least one location in the length direction of the rod portion 131. In the circumferential direction of the rod portion 131, as one example, the support hairs 132 may be disposed so as to be symmetrical around the rod portion 131 as shown in FIG. 7. By doing so, it is possible to stably support the temperature sensor 110 using the plurality of support hairs 132.

Also, although the plurality of support hairs 132 may be provided at at least one location in the length direction of the rod portion 131, that is a direction along the ernal auditory canal 12, to support the temperature sensor 110 more stably, it is possible to support the rod portion 131 at at least three locations to keep the rod portion 131 in the center of the ernal auditory canal 12. In this case, if the support hairs 132 are disposed along the entire length inside the ernal auditory canal 12 as shown in FIG. 16 for example, since the center of the sensor wire 120 is held at the contact points P1, P2, P3 between the support hairs 132 and the wall surface 12a of the ernal auditory canal 12, it is possible to keep the positions of the temperature sensor 110 and the sensor wire 120 inside the ernal auditory canal 12 in the center along the entire ernal auditory canal 12.

Also, as shown in FIG. 17, by locally disposing the support hairs 132 in the vicinity of the temperature sensor 110, it is possible to more reliably keep the temperature sensor 110 facing the eardrum 14 in an optimal state for measuring the radiant heat from the eardrum 14. At this time, the disposed spacing L of the support hairs 132 in the length direction of the rod portion 131 is short compared to the case shown in FIG. 16 so that the support hairs 132 are densely provided. It should be obvious that it is possible to combine the example arrangements of the support hairs 132 shown in FIG. 16 and FIG. 17 and that the arrangement of the support hairs 132 can be changed as appropriate.

By providing the plurality of support hairs 132 that bend upward and back toward the eardrum 14 on the sensor wire 120 inserted into the ernal auditory canal 12 as in the present embodiment, the front ends 132b of the support hairs 132 make point contact with the wall surface 12a of the ernal auditory canal 12 so that the support hairs 132 uniformly support the sensor wire 120. Also, since the support hairs 132 are an elastic material, the orientation and angle can freely change in keeping with the shape of the ernal auditory canal 12, which keeps the sensor wire 120 positioned in the center of the ernal auditory canal 12.

If the plurality of support hairs 132 are disposed at a plurality of locations in the length direction of the rod portion 131, to adapt to the shape of the ernal auditory canal 12 that narrows from the entrance toward the eardrum 14, the length of the support hairs 132 may be set so as to increase from the eardrum 14 side of the ernal auditory canal 12 toward the entrance. By doing so, it is possible to stabilize the supporting of the sensor wire 120 when the support hairs 132 contact the wall surface 12a of the ernal auditory canal 12. Accordingly, it is possible to fix the sensor wire 120 connected to the temperature sensor 110 stably at every position of the ernal auditory canal 12 without being dependent on the width of the ernal auditory canal 12. Also, even if the sensor wire 120 is moved inside the ernal auditory canal 12 in the radial direction perpendicular to the direction in which the ernal auditory canal 12 extends, due to the support hairs 132, the sensor wire 120 will then return to the center of the ernal auditory canal 12.

Also, by fixing the sensor wire 120 connected to the temperature sensor 110 using the plurality of support hairs 132 as in the present embodiment, a space that connects the outside of the ernal auditory canal 12 and the eardrum 14 will be present inside the ernal auditory canal 12. Accordingly, it will be possible to eradicate any turbulence due to the plurality of support hairs 132 and to enable sound to reach the eardrum 14, thereby improving usability.

In this way, by constructing the fixing mechanism of the temperature sensor 110 using the plurality of support hairs 132, it is possible to hold the temperature sensor 110 and the sensor wire 120 stably even when the temperature sensor 110 is inserted into the ernal auditory canal 12 for a long period.

### 3. Detection of Distance Between Eardrum and Temperature Sensor

The eardrum thermometer 100 is used by inserting the temperature sensor 110 inside the ernal auditory canal 12 as far as a position where the radiant heat from the eardrum 14 can be detected. Although contact between the temperature sensor 110 and the eardrum 14 is prevented with the eardrum thermometer 100 according to the present embodiment due to the provision of the fixing mechanism and the stopper, to further increase safety, it is also possible to equip the eardrum thermometer 100 with a distance detecting function that acquires the distance between the temperature sensor 110 and the eardrum 14. The distance detecting function that detects the distance between the temperature sensor 110 and the eardrum will now be described.

3-1. Overview of Distance Detecting Function

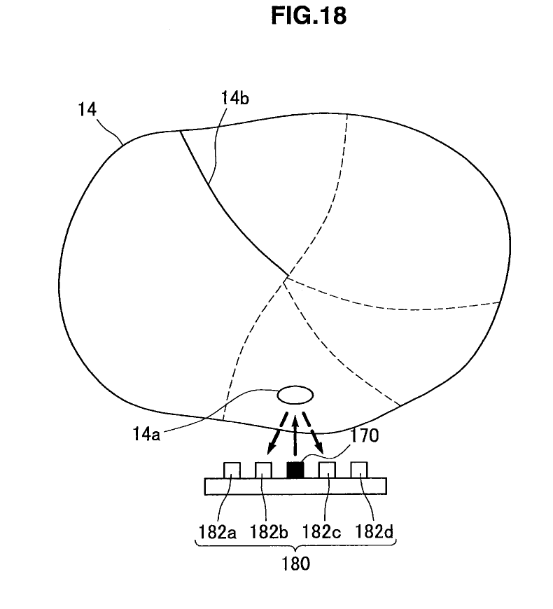

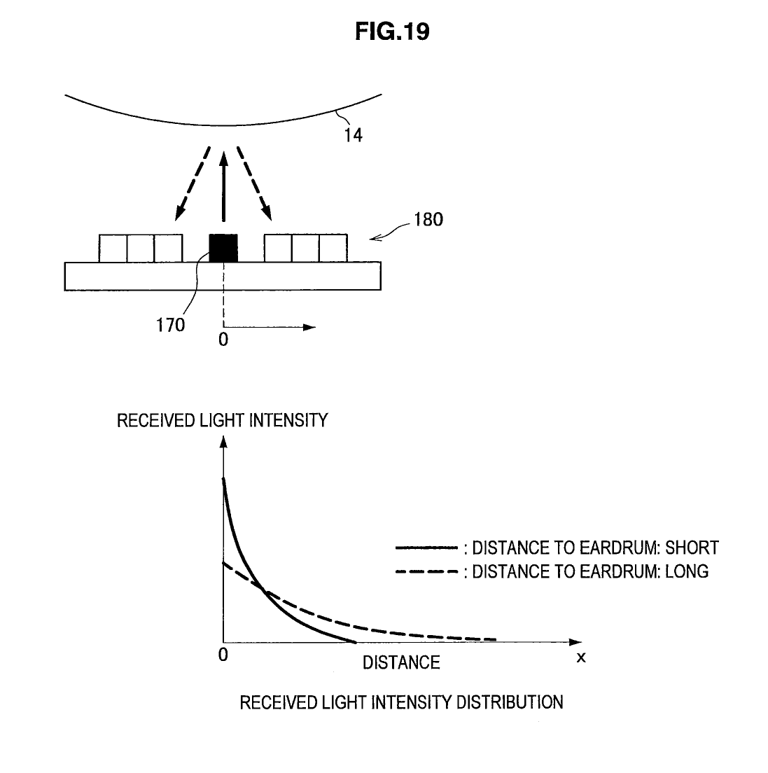

First, an overview of the distance detecting function will be described with reference to FIGS. 18 and 19. FIG. 18 is a diagram useful in explaining the relationship between the distance detecting sensor and the eardrum 14 according to the present embodiment. FIG. 19 is a diagram useful in explaining the positional relationship between a received light intensity distribution detected by the distance detecting sensor and the position of the distance detecting sensor relative to the eardrum.

With the distance detecting function according to the present embodiment, a light cone 14a that is a part of the eardrum 14 that appears to shine due to the reflection of external light is used to measure the distance to the eardrum 14. The light cone 14a is a phenomenon that is typically observed in a region on the opposite side of the center of the eardrum 14 to the manubrium mallei 14b during observation of the eardrum 14 by an ENT physician, and can be confirmed using an otoscope, an ear scope, or the like.

In the present embodiment, distance is measured by estimating the distance to the eardrum 14 using the reflected light produced when light is incident on the eardrum 14. As a surface that faces the ernal auditory canal 12 and favorably reflects light, the light cone 14a is suited to use when measuring the intensity of received light. As shown in FIG. 18, the distance detecting sensor is composed of a light emitting unit 170 that emits light onto the eardrum 14 and a light receiving unit 180 that receives reflected light for the light emitted from the light emitting unit 170. The reflection of light by the light cone 14a is believed to be the same as the reflection of light by the surface of a sphere. Here, if it is assumed that parallel light is emitted from the light emitting unit 170 onto the eardrum 14 from the center of the ernal auditory canal 12, the angle of reflection of light will increase as the distance from a vertex of the sphere (that is, the light cone 14a) increases. Accordingly, the light reflected by the light cone 14a is scattered.

The light reflected by the light cone 14a is received by the light receiving unit 180. Since the reflected light is scattered as described above, the light receiving unit 180 according to the present embodiment is constructed with a plurality of light-receiving elements 182 (in FIG. 18, light-receiving elements 182a to 182d) in a one-dimensional array or a two-dimensional array. By doing so, it is possible to increase the light receiving range of the scattered reflected light and to also acquire a received light intensity distribution showing the received light intensity for each light-receiving element 182. Note that the light-receiving elements 182 that construct the light receiving unit 180 may be disposed as shown in FIG. 18 for example at an equal spacing centered on and around the periphery of the light emitting unit 170.

The light receiving range of the light receiving unit 180 for the reflected light changes according to the distance (hereinafter referred to as “light-receiving distance”) between the eardrum 14 and the light receiving unit 180. That is, when the light-receiving distance is comparatively short, since the range in which light is scattered by the light cone 14a is narrow, as shown by the solid line at the bottom of FIG. 19, the received light intensity at the position (x=0) that is closest to the light cone 14a is high and the light receiving range has a narrow distribution. Meanwhile, when the light-receiving distance is comparatively long, since the range in which light is scattered by the light cone 14a is wide, as shown by the broken line at the bottom of FIG. 19, the received light intensity at the position (x=0) that is closest to the light cone 14a is not especially high and the light receiving range has a wide distribution.

The distance detecting function according to the present embodiment estimates the distance between the eardrum 14 and the distance detecting sensor based on the result of such received light intensity distribution.

3-2. Functional Configuration of Distance Detecting Apparatus

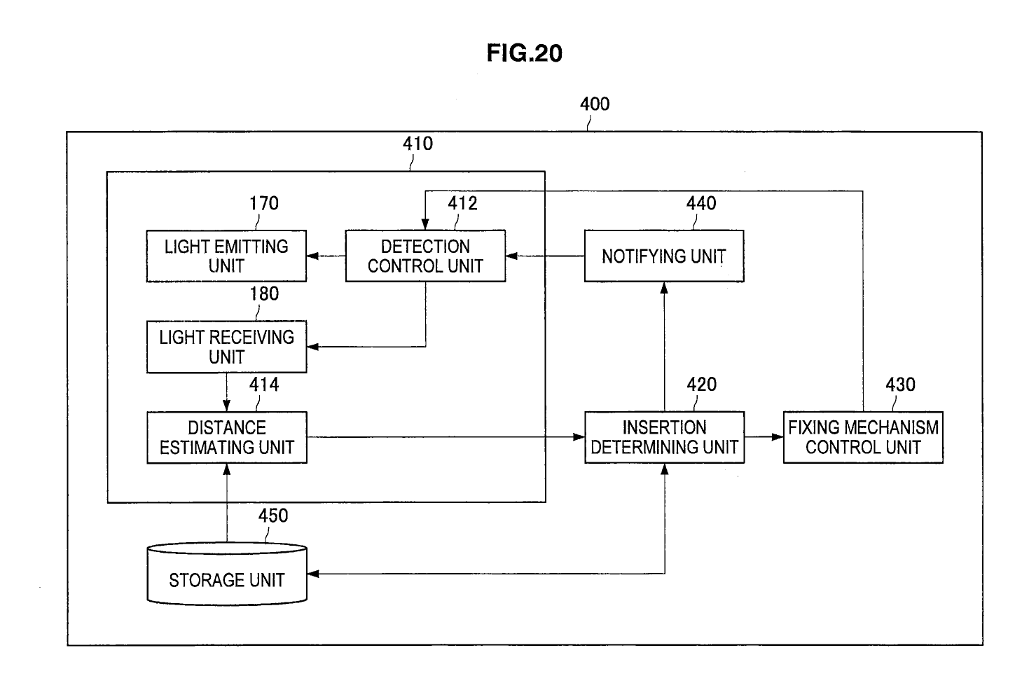

Next, a distance detecting apparatus 400 that functions as the distance detecting function will be described with reference to FIG. 20. FIG. 20 is a functional block diagram showing the functional configuration of the distance detecting apparatus 400 according to the present embodiment.

As shown in FIG. 20, the distance detecting apparatus 400 according to the present embodiment includes a distance detecting unit 410, an insertion determining unit 420, a fixing mechanism control unit 430, a notifying unit 440, and a storage unit 450.

The distance detecting unit 410 estimates the distance from the eardrum 14 to the temperature sensor 110 based on the received light intensity distribution produced by the distance detecting sensor. The distance detecting unit 410 includes the light emitting unit 170 and the light receiving unit 180 that are the distance detecting sensor, a detection control unit 412, and a distance estimating unit 414. The light emitting unit 170 emits light onto the eardrum 14. The light emitting unit 170 can be constructed from a light emitting element, such as an LED. The light receiving unit 180 receives the light reflected by the eardrum 14. The light receiving unit 180 is constructed by aligning a plurality of light-receiving elements 182 such as photodiodes. The light receiving unit 180 outputs the received light intensities detected by the respective light-receiving elements 182 to the distance estimating unit 414.

The detection control unit 412 controls the light emitting unit 170 and the light receiving unit 180 that are the distance detecting sensor. Based on a distance detection start instruction from the user or the eardrum thermometer 100, the detection control unit 412 instructs the light emitting unit 170 to emit light and instructs the light receiving unit 180 to receive the light reflected by the eardrum 14.

The distance estimating unit 414 generates a received light intensity distribution based on the received light intensities detected by the respective light-receiving elements 182 inputted from the light receiving unit 180 and estimates the distance from the eardrum 14 to the distance detecting sensor. As shown in FIG. 19, the distance estimating unit 414 calculates the distance from the eardrum 14 to the distance detecting sensor based on the form of the received light intensity distribution produced by the light receiving unit 180, that is, the intensity and spatial diffusion of the light received by the eardrum 14. The correspondence between distance from the eardrum 14 to the distance detecting sensor and the received light intensity distribution is acquired in advance and is stored in the storage unit 450, described later. Based on the received light intensity distribution acquired by a present detection operation of the light receiving unit 180, The distance estimating unit 414 estimates the light-receiving distance from the correspondence between the distance from the eardrum 14 to the distance detecting sensor stored in the storage unit 450 and the received light intensity distribution. The estimated light-receiving distance is outputted to the insertion determining unit 420.

The insertion determining unit 420 determines whether the temperature sensor 110 may move toward the eardrum 14 based on the light-receiving distance. The insertion determining unit 420 compares a threshold distance stored in the storage unit 450 and the light-receiving distance (or eardrum to temperature sensor distance) and determines whether the temperature sensor 110 may move toward the eardrum 14. The determination result of the insertion determining unit 420 is outputted to the fixing mechanism control unit 430 and the notifying unit 440.

The fixing mechanism control unit 430 controls the fixing mechanism of the eardrum thermometer 100 based on the determination result of the insertion determining unit 420. That is, if the insertion determining unit 420 has determined that insertion of the temperature sensor 110 is to be stopped, the fixing mechanism control unit 430 operates the fixing mechanism to place the support hairs in an erect state so that the temperature sensor 110 is fixed in the ernal auditory canal 12 so as to not move. Meanwhile, if the insertion determining unit 420 has determined that the stopping of insertion of the temperature sensor 110 is to be released, the fixing mechanism control unit 430 operates the fixing mechanism to place the support hairs in the reclining state so that the temperature sensor 110 becomes capable of moving inside the ernal auditory canal 12. When operating the fixing mechanism in accordance with the determination result of the insertion determining unit 420, the fixing mechanism control unit 430 notifies the detection control unit 412 of such operation.

Note that the fixing mechanism controlled by the fixing mechanism control unit 430 may use the support hairs described above and shown in FIG. 1, for example, or may be a fixing mechanism of a different construction. As another example, the fixing mechanism may be an expanding member, such as a balloon, provided on the sensor wire 120 that is connected to the temperature sensor 110 and extends toward the entrance of the ernal auditory canal 12, the expanding member expanding inside the ernal auditory canal 12 so as to apply pressure to the wall surface 12a of the ernal auditory canal 12.

The notifying unit 440 notifies the user of the determination result of the insertion determining unit 420. As examples, notification of the determination result of the insertion determining unit 420 may be given by way of sound and vibration, the color of light, a light emission pattern, and the like. Accordingly, the notifying unit 440 is capable of being constructed of at least one of an audio output unit such as a speaker or bone-conduction device that outputs audio, a vibration generation unit that generates vibration, a light-emitting unit such as a LED, and a display unit. It should be obvious that the apparatus for giving notification of the determination result can also be used as the notifying unit 440. On notifying the user of the determination result of the insertion determining unit 420, the notifying unit 440 notifies the detection control unit 412.

The storage unit 450 stores information used by the distance detecting function. The storage unit 450 stores the correspondence between the distance from the eardrum 14 to the distance detecting sensor and the light receiving intensity distribution, threshold distances, and the like. Also, if there is a difference in distance between the distance detecting sensor and the temperature sensor 110, such information is also stored in the storage unit 450. By correcting the light-receiving distance according to such difference in distance, it is possible to calculate the distance between the eardrum 14 and the temperature sensor 110 (the eardrum to temperature sensor distance).

3-3. Navigation by Distance Detecting Apparatus

The distance detecting apparatus 400 according to the present embodiment detects the distance (i.e., the eardrum to temperature sensor distance) between the eardrum 14 and the temperature sensor 110. At this time, the distance detecting apparatus 400 determines whether it is possible to insert the eardrum thermometer 100 toward the eardrum 14 based on the eardrum to temperature sensor distance. Based on the determination result, the distance detecting apparatus 400 operates the fixing mechanism and notifies the user of the determination result. By doing so, it is possible for the user to move the temperature sensor 110 while confirming the insertion state of the temperature sensor 110 using the distance detecting apparatus 400. Also, since it is decided whether the temperature sensor 110 can be moved in accordance with the inserted state of the temperature sensor 110 and the fixing mechanism is operated automatically, even if the user carries out an operation that contradicts the determination result, the temperature sensor 110 does not move when such an operation is performed. In this way, the distance detecting apparatus 400 according to the present embodiment is capable of carrying out navigation that moves the temperature sensor 110 safely.

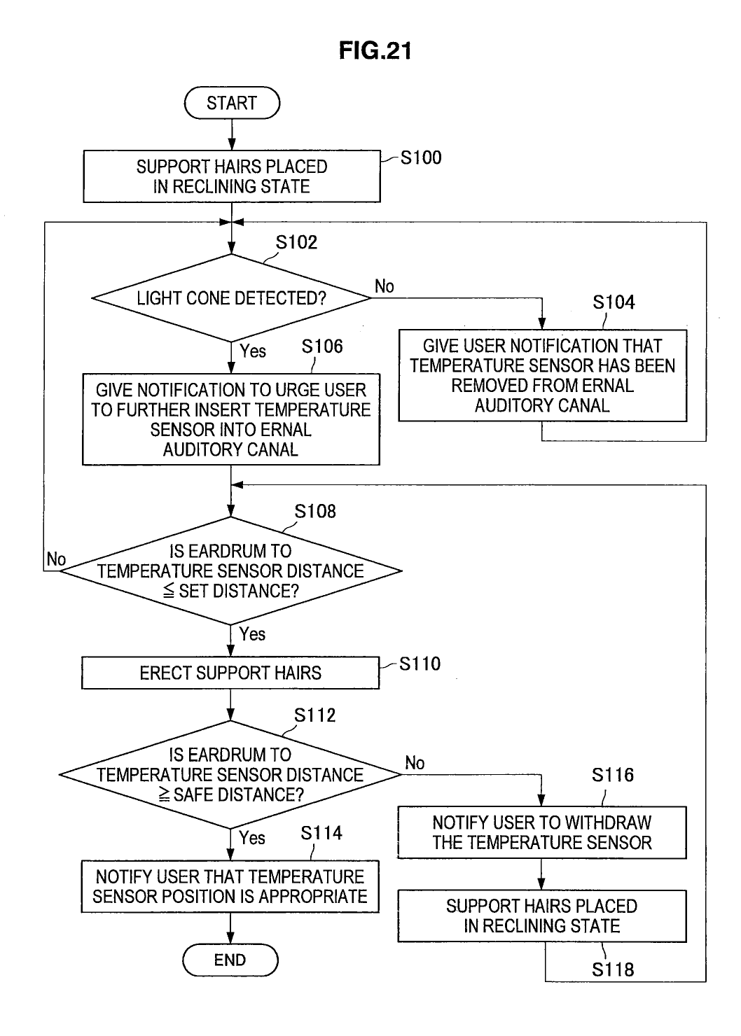

Next, a navigation process carried out by the distance detecting apparatus 400 will be described with reference to FIG. 21. FIG. 21 is a flowchart showing the navigation process carried out by the distance detecting apparatus 400 according to the present embodiment.

When the temperature sensor 110 of the eardrum thermometer 100 is inserted inside the ernal auditory canal 12, the support hairs are placed in the reclining state (S100). When the temperature sensor 110 is inserted inside the ernal auditory canal 12, as one example it is determined by the insertion determining unit 420 whether the light cone 14a has been detected at specified time intervals (S102). It is possible to determine whether the light cone 14a has been detected according to the received light intensity detected by the light receiving unit 180. If light is incident on the light cone 14a, a received light intensity of a specified intensity or higher will be detected. For this reason, the insertion determining unit 420 determines whether the highest intensity out of the received light intensity detected by the light-receiving elements 182 that construct the light receiving unit 180 is a specified intensity or higher.

If the highest received light intensity is smaller than the specified intensity in step S102, the insertion determining unit 420 determines that the temperature sensor 110 has been removed from the ernal auditory canal 12 and instructs the notifying unit 440 to notify the user of such determination result (S104). The user who has received such notification then corrects the insertion direction of the temperature sensor 110. The distance detecting apparatus 400 repeats the processing from step S102. Meanwhile, if the highest received light intensity is equal to or higher than the specified intensity in step S102, it is determined that the temperature sensor 110 is correctly inserted in the ernal auditory canal 12 and the insertion determining unit 420 instructs the notifying unit 440 to give notification to urge the user to further insert the temperature sensor 110 (S106).

After this, the insertion determining unit 420 determines whether the eardrum to temperature sensor distance that is the distance between the eardrum 14 and the temperature sensor 110 is equal to or below a set distance (S108). The set distance is set as a distance where it is possible to measure radiant heat from the eardrum 14 and is stored in the storage unit 450 as one of the threshold distances. The insertion determining unit 420 corrects the light receiving distance calculated by the distance estimating unit 414 for the difference in distance between the distance detecting sensor and the temperature sensor 110 to calculate the eardrum to temperature sensor distance. After this, if the eardrum to temperature sensor distance is not equal to or shorter than the set distance, the insertion determining unit 420 determines that the temperature sensor 110 may be moved closer to the eardrum 14 and the processing is repeated from step S102.

Meanwhile, if the eardrum to temperature sensor distance is equal to or shorter than the set distance, the insertion determining unit 420 instructs the fixing mechanism control unit 430 to erect the support hairs to stop the temperature sensor 110 (S110). The insertion determining unit 420 then determines whether the eardrum to temperature sensor distance is equal to or above a safe distance (S112). The safe distance is set at the shortest distance where the temperature sensor 110 does not contact the eardrum 14 and is stored in the storage unit 450 as one of the threshold distances. If the eardrum to temperature sensor distance is equal to or above the safe distance, the insertion determining unit 420 determines that the temperature sensor 110 is at an appropriate position to measure the radiant heat from the eardrum 14 and instructs the notifying unit 440 to notify the user (S114) and the processing in FIG. 21 ends.

If it is determined in step S112 that the eardrum to temperature sensor distance is shorter than the safe distance, it is determined that there is the risk of the temperature sensor 110 contacting the eardrum 14. In this case, the insertion determining unit 420 instructs the notifying unit 440 to notify the user to withdraw the temperature sensor 110 (S116) and instructs the fixing mechanism control unit 430 to erect the support hairs (S118). After this, the processing is repeated from step S108.

The configuration and functions of the distance detecting apparatus 400 that can be adapted to the eardrum thermometer 100 according to the present embodiment have been described above. The distance detecting apparatus 400 is capable of having the distance detecting sensor provided in the vicinity of the temperature sensor 110 of the eardrum thermometer 100 and a function unit that processes the detection result of the distance detecting sensor provided outside the ernal auditory canal 12. The function unit that is provided outside the ernal auditory canal 12 is configured so as to be capable of communicating with the distance detecting sensor. The distance estimating unit 414 of the distance detecting apparatus 400 is capable of calculating the light receiving distance based on the received light intensity distribution. In this way, by acquiring information inside the ernal auditory canal 12 to acquire a more accurate light-receiving distance, it is possible to notify the user of the distance between the eardrum 14 and the temperature sensor 110 and to use the temperature sensor 110 safely.

Also, by providing the navigation function of the distance detecting apparatus 400, it is possible to guide the insertion of the temperature sensor 110 until an appropriate position for measuring the radiant heat from the eardrum 14 is reached and to operate the fixing mechanism when the appropriate position has been reached to fix the temperature sensor 110. In addition, if the temperature sensor 110 has moved too close to the eardrum 14, the distance detecting apparatus 400 gives notification to urge the user to perform an operation to move the temperature sensor 110 back toward the entrance of the ernal auditory canal 12. By using this navigation function, it is possible for the user to easily and safely use the temperature sensor 110.

### 4. Example Hardware Configuration

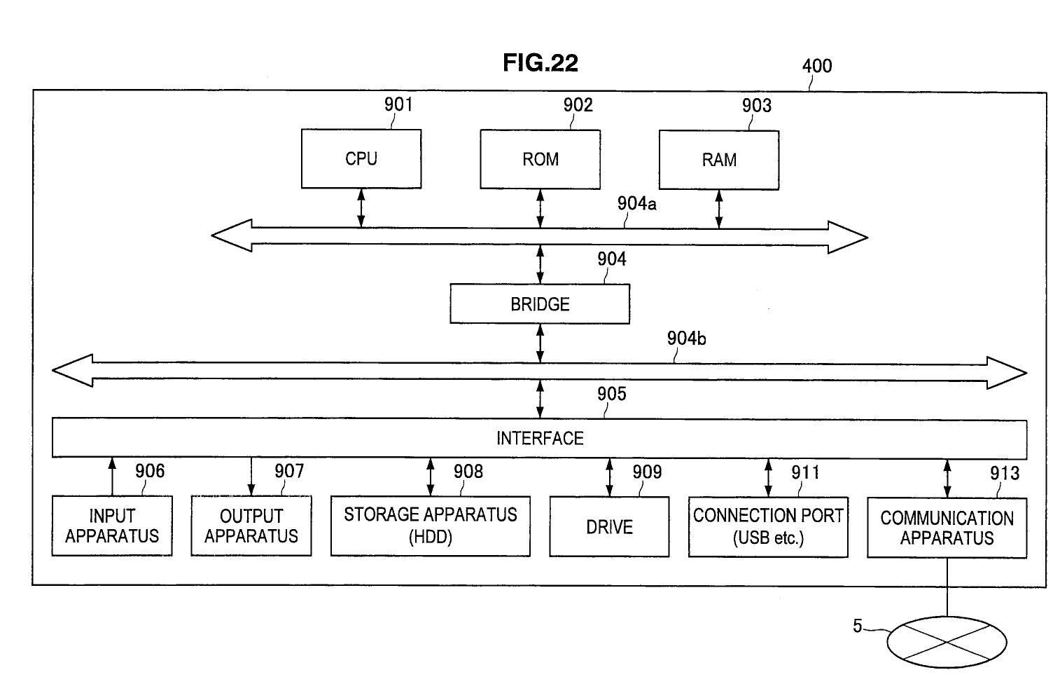

The processing carried out by the distance detecting apparatus 400 according to the present embodiment can be executed by hardware and can also be executed by software. In this case, the distance detecting apparatus 400 can be configured as shown in FIG. 22. An example of the hardware configuration of the distance detecting apparatus 400 according to the present embodiment will now be described with reference to FIG. 22.

As described earlier, the distance detecting apparatus 400 according to the present embodiment can be realized by a processing apparatus such as a computer. As shown in FIG. 22, the distance detecting apparatus 400 includes a CPU (Central Processing Unit) 901, a ROM (Read Only Memory) 902, a RAM (Random Access Memory) 903 and a host bus 904a. The distance detecting apparatus 400 also includes a bridge 904, an external bus 904b, an interface 905, an input apparatus 906, an output apparatus 907, a storage apparatus (HDD) 908, a drive 909, a connection port 911, and a communication apparatus 913.

The CPU 901 functions as a computational processing apparatus and a control apparatus and controls the overall operation inside the distance detecting apparatus 400 in accordance with various programs. The CPU 901 may be a microprocessor. The ROM 902 stores programs, various computation parameters, and the like used by the CPU 901. The RAM 903 temporarily stores programs used for execution by the CPU 901, parameters that change as appropriate during such execution, and the like. Such components are connected to one another by the host bus 904a constructed of a CPU bus or the like.

The host bus 904a is connected via the bridge 904 to the external bus 904b which is a PCI (Peripheral Component Interconnect/Interface) bus or the like. Note that the host bus 904a, the bridge 904, and the external bus 904b do not need to be constructed separately and such functions may be implemented by a single bus.

The input apparatus 906 includes an input device, such as a mouse, a keyboard, a touch panel, a button or buttons, a microphone, a switch or switches, and a lever or levers, which enables the user to input information and an input control circuit that generates an input signal based on an input made by the user and outputs the input signal to the CPU 901. As examples, the output apparatus 907 includes a display apparatus such as a liquid crystal display (LCD) apparatus, an OLED (Organic Light Emitting Diode) apparatus, and a lamp or lamps, and an audio output apparatus such as a speaker.

The storage apparatus 908 is one example of a storage unit of the distance detecting apparatus 400 and is an apparatus for storing data. The storage apparatus 908 may include a storage medium, a recording apparatus that records data onto a storage medium, a read apparatus that reads data from a storage medium, a deletion apparatus that deletes data recorded on a storage medium, and the like. The storage apparatus 908 is constructed of an HDD (Hard Disk Drive) for example. This storage apparatus 908 drives a hard disk and stores a program executed by the CPU 901 and various data.

The drive 909 is a reader/writer for a storage medium and is built into or externally attached to the distance detecting apparatus 400. The drive 909 reads information recorded on a removable storage medium, such as a magnetic disk, an optical disc, a magneto-optical disc, or a semiconductor memory, that has been loaded and outputs to the RAM 903.

The connection port 911 is an interface connected to an external appliance and is a connection port for an external appliance that is capable of data transfer using USB (Universal Serial Bus), for example. The communication apparatus 913 is a communication interface constructed by a communication device or the like for connecting to a communication network 5, for example. Also, the communication apparatus 913 may be a wireless LAN (Local Area Network)-compliant communication apparatus, a wireless USB-compliant communication apparatus, or a wired communication apparatus that carries out communication using wires.

Although preferred embodiments of the present disclosure have been described above in detail with reference to the attached drawings, the technical scope of the present disclosure is not limited to such embodiments. It should be understood by those skilled in the art that various modifications, combinations, sub-combinations and alterations may occur depending on design requirements and other factors insofar as they are within the scope of the appended claims or the equivalents thereof.

Additionally, the present technology may also be configured as below.

(1) An in-ear information acquiring apparatus comprising:

an information acquiring unit that is inserted in the ernal auditory canal and acquires information inside the ear; and

a fixing mechanism that fixes the information acquiring unit inside the ernal auditory canal,

wherein the fixing mechanism includes:

a rod portion having a plurality of support portions, which are erected toward a wall surface of the ernal auditory canal, provided on an outer side surface thereof around an outer circumference thereof at at least one location along the ernal auditory canal; and

an adjusting portion adjusting an erect state of the support portions provided on the rod portion.

(2) The in-ear information acquiring apparatus according to (1),

wherein the rod portion covers a wire that is connected to the information acquiring unit and extends toward an entrance of the ernal auditory canal.

(3) The in-ear information acquiring apparatus according to (1) or (2),

wherein the plurality of support portions, which are erected toward the wall surface of the ernal auditory canal, are provided on the outer side surface of the rod portion around an outer circumference of the rod portion at a plurality of locations along the ernal auditory canal.

(4) The in-ear information acquiring apparatus according to any one of (1) to (3),

wherein a length of each support portion is set longer the closer the disposed position of each support portion toward the entrance to the ernal auditory canal from the information acquiring unit side.

(5) The in-ear information acquiring apparatus according to any one of (1) to (4),

wherein the adjusting portion includes:

a plurality of ring-shaped members through which the support portions are passed; and

a ring-shaped operation portion that operates positions of the ring-shaped members,

wherein the ring-shaped operation portion moves the ring-shaped members along a length direction of the rod portion.

(6) The in-ear information acquiring apparatus according to any one of (1) to (4),

wherein the adjusting portion includes:

a plurality of ring-shaped members through which the support portions are passed; and

a ring-shaped operation portion that operates positions of the ring-shaped members,

wherein the ring-shaped operation portion rotationally moves the ring-shaped members in a circumferential direction of the rod portion.

(7) The in-ear information acquiring apparatus according to any one of (1) to (6),

wherein the information acquiring unit is a temperature sensor measuring radiant heat from the eardrum.

(8) The in-ear information acquiring apparatus according to (7),

wherein the information acquiring unit includes a distance detecting sensor detecting distance from the eardrum to the information acquiring unit.

(9) The in-ear information acquiring apparatus according to (8),

wherein the distance detecting sensor includes a light emitting unit emitting light onto the eardrum and a light receiving unit receiving light reflected by the eardrum.

(10) The in-ear information acquiring apparatus according to (9),

wherein the light receiving unit receives the reflected light from a light cone.

(11) A fixing mechanism comprising:

a rod portion having a plurality of support portions, which are erected toward a wall surface of the ernal auditory canal, provided on an outer side surface thereof around an outer circumference thereof at at least one location along the ernal auditory canal; and

an adjusting portion adjusting an erect state of the support portions provided on the rod portion.

The present disclosure contains subject matter related to that disclosed in Japanese Priority Patent Application JP 2011-170033 filed in the Japan Patent Office on Aug. 3, 2011, the entire content of which is hereby incorporated by reference.

What is claimed is:

1. An in-ear information acquiring apparatus comprising:
an information acquiring unit that is inserted in the ernal auditory canal and acquires information inside the ear; and
a fixing mechanism that fixes the information acquiring unit inside the ernal auditory canal,
wherein the fixing mechanism includes:
a rod portion having a plurality of support portions, which are erected toward a wall surface of the ernal auditory canal, provided on an outer side surface thereof around an outer circumference thereof at at least one location along the ernal auditory canal; and
an adjusting portion adjusting an erect state of the support portions provided on the rod portion.

2. The in-ear information acquiring apparatus according to claim 1,
wherein the rod portion covers a wire that is connected to the information acquiring unit and extends toward an entrance of the ernal auditory canal.

3. The in-ear information acquiring apparatus according to claim 1,
wherein the plurality of support portions, which are erected toward the wall surface of the ernal auditory canal, are provided on the outer side surface of the rod portion around an outer circumference of the rod portion at a plurality of locations along the ernal auditory canal.

4. The in-ear information acquiring apparatus according to claim 1,
wherein a length of each support portion is set longer the closer the disposed position of each support portion toward the entrance to the ernal auditory canal from the information acquiring unit side.

5. The in-ear information acquiring apparatus according to claim 1,
wherein the adjusting portion includes:
a plurality of ring-shaped members through which the support portions are passed; and
a ring-shaped operation portion that operates positions of the ring-shaped members,
wherein the ring-shaped operation portion moves the ring-shaped members along a length direction of the rod portion.

6. The in-ear information acquiring apparatus according to claim 1,
wherein the adjusting portion includes:
a plurality of ring-shaped members through which the support portions are passed; and
a ring-shaped operation portion that operates positions of the ring-shaped members,
wherein the ring-shaped operation portion rotationally moves the ring-shaped members in a circumferential direction of the rod portion.

7. The in-ear information acquiring apparatus according to claim 1,
wherein the information acquiring unit is a temperature sensor measuring radiant heat from the eardrum.

8. The in-ear information acquiring apparatus according to claim 7,
wherein the information acquiring unit includes a distance detecting sensor detecting distance from the eardrum to the information acquiring unit.

9. The in-ear information acquiring apparatus according to claim 8,
wherein the distance detecting sensor includes a light emitting unit emitting light onto the eardrum and a light receiving unit receiving light reflected by the eardrum.

10. The in-ear information acquiring apparatus according to claim 9,
wherein the light receiving unit receives the reflected light from a light cone.

11. A fixing mechanism comprising:
a rod portion having a plurality of support portions, which are erected toward a wall surface of the ernal auditory canal, provided on an outer side surface thereof around an outer circumference thereof at at least one location along the ernal auditory canal; and
an adjusting portion adjusting an erect state of the support portions provided on the rod portion.
